(19) United States
(12) Patent Application Publication
(10) Pub. No.: US 2024/0358631 A1
(43) Pub. Date: Oct. 31, 2024

(54) TOPICAL FORMULATIONS

(71) Applicant: ProVEDA Corporation, Miramar Beach, FL (US)

(72) Inventors: Clyde Rowland HANSON, Miramar Beach, FL (US); Ronnie Scott GRIZZLE, Miramar Beach, FL (US)

(21) Appl. No.: 18/307,253

(22) Filed: Apr. 26, 2023

(51) Int. Cl. A61K 9/00; A61K 9/06; A61K 31/00; A61K 47/44; A61P 25/04; A61P 29/00

(52) U.S. Cl. CPC A61K 9/0014; A61K 9/06; A61K 31/658; A61K 47/44; A61P 25/04; A61P 29/00

(57) ABSTRACT

The present disclosure provides a topical formulation that possesses analgesic and anti-inflammatory activity. An aspect of the present disclosure provides a topical formulation including: (a) an essential oil in an amount ranging from 10% by wt. to 45% by wt.; (b) an active ingredient in an amount ranging from 2% by wt. to 10% by wt. and (c) an excipient. It further relates to a method of preparation of topical formulation in the form of a cream. It further relates to a method of reducing pain and inflammation in a mammal resulting from muscle strain, spasms, arthritis, and bursitis associated with sports related injury by topical administration of the formulation.

### FIELD OF THE INVENTION

The present invention relates generally to the field of topical formulations. Particularly, the present disclosure provides a topical formulation that possesses analgesic and anti-inflammatory activity. It further relates to a method of reducing pain and inflammation in a mammal resulting from muscle strain, spasms, arthritis, and bursitis associated with sports related injury by topical administration of the formulation. It further relates to a method of preparation of topical formulation in the form of a cream.

### BACKGROUND OF THE INVENTION

Background description includes information that may be useful in understanding the present invention. It is not an admission that any of the information provided herein is prior art or relevant to the presently claimed invention, or that any publication specifically or implicitly referenced is prior art.

Administration of therapeutic agents either locally to the skin or into the systemic circulation after passage through the skin, offers numerous potential advantages over oral, parenteral and intravenous drug delivery systems. It includes avoidance of hepatic first-pass metabolism, improved patient compliance and ease of access to the absorbing membrane, i.e. the skin. Further, in case of local delivery (i.e. delivery to the superficial layers of the skin) by directly administering the therapeutic agent to the requisite site (pathological or pain), any adverse effects associated with systemic toxicity can also be minimized. Currently available topical formulations are reported to have drawbacks resulting in limited penetration and low bioavailability of active molecules affecting the efficacy of the product along with dermal safety concerns.

There is therefore an unmet need in the art to develop new and improved topical formulations that may overcome the drawbacks associated with the existing topical formulations. The present invention satisfies the existing needs, as well as others, and generally overcomes the deficiencies found in the prior art.

### SUMMARY OF THE INVENTION

The present disclosure relates generally to the field of topical formulations. Particularly, the present disclosure provides a topical formulation that possesses analgesic and anti-inflammatory activity. It further relates to a method of preparation of topical formulation in the form of a cream.

An aspect of the present disclosure provides a topical formulation including: (a) one or more essential oils in an amount ranging from 10% by wt. to 45% by wt.; (b) one or more active ingredients in an amount ranging from 2% by wt. to 10% by wt. and (c) one or more excipients. In an embodiment, the essential oil includes spearmint oil, eucalyptus oil, peppermint oil, clove bud oil, thyme oil, capsicum oleoresin oil, lavender oil, rosemary oil, myrrh oil, ginger oil, orange sweet oil, chamomile oil, wintergreen oil, ajwain oil and combinations thereof. In an embodiment, the excipient includes: an emulsifier, a pH modifying agent, a solvent, a gelling agent and combinations thereof. In an embodiment, the active ingredient includes: an aqueous extract of chaste tree, an aqueous extract of guggul, an aqueous extract of triglyphix sense, menthol, camphor and combinations thereof. In an embodiment, the topical formulation is a cream formulation.

In an embodiment, the cream formulation has firmness (peak positive force at cycle 1) ranging from 140 g to 180 g. In an embodiment, the cream formulation has stickiness (peak negative force at cycle 1) ranging from −150 g to −190 g. In an embodiment, the cream formulation has work of shear (Area F-T 1:2) ranging from 160 g·sec to 190 g·sec. In an embodiment, the cream formulation has index of viscosity (Area F-T 3:4) ranging from −20 g·sec to −50 g·sec. In an embodiment, the cream formulation has a penetration rate (flux) of at least 12 μg/(cm2*h) when measured using Franz diffusion cell mounted with a Strat-M membrane. In an embodiment, the cream formulation has a penetration rate (flux) of at least 15 μg/(cm2*h) when measured using Franz diffusion cell mounted with a Strat-M membrane. In an embodiment, the cream formulation has a penetration rate (flux) of at least 20 μg/(cm2*h) when measured using Franz diffusion cell mounted with a Strat-M membrane. In an embodiment, the cream formulation has a penetration rate (flux) ranging from 20 to 27 μg/(cm2*h) when measured using Franz diffusion cell mounted with a Strat-M membrane. In an embodiment, the cream formulation has ex-vivo skin penetration of at least 200 μm when measured on Guinea pig skin using confocal microscopy, preferably, at least 250 μm, and more preferably, at least 300 μm.

In an embodiment, the cream formulation further includes a particulate stock formulation as a natural analgesic lipid phase in an amount ranging from 5% by wt. to 75% by wt., said stock formulation including: a lipid phase, natural analgesic phase, a solubilizer and a surfactant solution, wherein said natural analgesic is different from said active ingredient and said essential oil. In an embodiment, the particulate stock formulation is a solid lipid nanoparticulate stock formulation.

Another aspect of the present disclosure relates to a method of preparation of a cream formulation, said method including the steps of: (a) taking essential oils and oil soluble ingredients to preparing an oil phase; (b) mixing active ingredient(s) and water soluble ingredients with water to prepare an aqueous solution; (c) mixing a gelling agent with the aqueous solution to prepare a water phase; (d) mixing a particulate stock formulation with the water phase to prepare a mixture; (e) adding the oil phase to the mixture from step (d) with stirring to prepare a crude emulsion; (f) adding an emulsifying agent and a pH modifying agent to the crude emulsion to prepare a coarse emulsion; and (g) homogenizing the coarse emulsion to prepare the cream formulation.

### BRIEF DESCRIPTION OF THE ACCOMPANYING DRAWINGS

The following drawings form part of the present specification and are included to further illustrate aspects of the present disclosure. The disclosure may be better understood by reference to the drawings in combination with the detailed description of the specific embodiments presented herein.

FIGS. 1A and 1B illustrate a snippet depicting confocal microscopic images of rat skin treated with placebo (Depth: 20 μm) and cream formulation (Depth: 185 μm), in accordance with the embodiments of the present disclosure.

FIG. 2A through 2D illustrate a snippet depicting confocal microscopic image of guinea pig skin treated with cream formulation (Depth: 330 μm), Charlotte's web (Depth: 150 μm), CBD Medic (Depth: 135 μm) and Myaderm (Depth: 60 μm), in accordance with the embodiments of the present disclosure.

FIG. 3 illustrates a graph depicting % inhibition of COX-2 activity for the cream formulation, realized in accordance with the embodiments of the present disclosure.

### DESCRIPTION OF THE PRESENT INVENTION

The embodiments herein and the various features and advantageous details thereof are explained more comprehensively with reference to the non-limiting embodiments that are detailed in the following description. Descriptions of well-known components and processing techniques are omitted so as to not unnecessarily obscure the embodiments herein. The examples used herein are intended merely to facilitate an understanding of the ways in which the embodiments herein may be practiced and to further enable those of skill in the art to practice the embodiments herein. Accordingly, the examples should not be construed as limiting the scope of the embodiments herein.

Unless otherwise specified, all terms used in disclosing the invention, including technical and scientific terms, have the meaning as commonly understood by one of ordinary skills in the art to which this invention belongs. By means of further guidance, term definitions may be included to better appreciate the teaching of the present invention.

As used in the description herein, the meaning of “a,” “an,” and “the” includes plural reference unless the context clearly dictates otherwise. Also, as used in the description herein, the meaning of “in” includes “in” and “on” unless the context clearly dictates otherwise.

As used herein, the terms “comprise”, “comprises”, “comprising”, “include”, “includes”, and “including” are meant to be non-limiting, i.e., other steps and other ingredients which do not affect the end of result can be added. The above terms encompass the terms “consisting of” and “consisting essentially of”.

As used herein, the terms “composition”, “formulation”, “blend”, or “mixture” are all intended to be used interchangeably.

The terms “weight percent”, “vol-%”, “percent by weight”, “% by weight”, and variations thereof, as used herein, refer to the concentration of a substance as the weight of that substance divided by the total weight of the composition and multiplied by 100. It is understood that, as used here, “percent”, “%”, and the like are intended to be synonymous with “weight percent”, “vol-%”, etc.

The term ‘solid lipid nanoparticulate’ as used herein, refers to a colloidal drug delivery system, composed of lipids, particularly, the physiological lipids that remain in a solid state, at both room and body temperature. Solid lipid nanoparticulate possesses a solid lipid core matrix that solubilizes the lipophilic molecules. Lipid core is stabilized by surfactants (emulsifiers) that depend on the routes of administrations. The term lipid is used here in a broader sense and includes triglycerides, diglycerides, monoglycerides, fatty acids, steroids and waxes. Solid lipid nanoparticulates are in the size range of 10 to 1000 nm.

The term “treatment” as used herein covers any treatment of a disease in a mammal, particularly a human, and includes:
(i) preventing the disease from occurring in a subject which may be predisposed to the disease but has not yet been diagnosed as having it;
(ii) inhibiting the disease, i.e. arresting its development; and
(iii) relieving the disease, i.e. causing regression of the disease.

The term “therapeutically effective amount” refers to that amount which is sufficient to effect treatment, as defined above, when administered to a mammal in need of such treatment. The therapeutically effective amount will vary depending on the subject and disease state being treated, the severity of the affliction and the manner of administration, and may be determined routinely by one of ordinary skill in the art. Thus, a therapeutically amount of an analgesic topical cream composition is that amount which is sufficient to effect treatment, as defined above, when administered to a mammal in need of treatment by an analgesic topical cream composition. A therapeutically amount of an anti-inflammatory topical cream composition is that amount which is sufficient to effect treatment, as defined above, when administered to a mammal in need of treatment by an anti-inflammatory topical cream composition.

In some embodiments, the numbers expressing quantities of ingredients, properties such as concentration, reaction conditions, and so forth, used to describe and claim certain embodiments of the invention are to be understood as being modified in some instances by the term “about”. Accordingly, in some embodiments, the numerical parameters set forth in the written description are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable.

The recitation of ranges of values herein is merely intended to serve as a shorthand method of referring individually to each separate value falling within the range. Unless otherwise indicated herein, each individual value is incorporated into the specification as if it were individually recited herein.

The headings and abstract of the invention provided herein are for convenience only and do not interpret the scope or meaning of the embodiments.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

The present disclosure relates generally to the field of topical formulations. Particularly, the present disclosure provides a topical formulation that may find its utility as an analgesic and an anti-inflammatory agent. It further relates to a method of preparation of topical formulation in the form of a cream.

An aspect of the present disclosure provides a topical formulation including: (a) one or more essential oils in an amount ranging from 10% by wt. to 45% by wt.; (b) one or more active ingredients in an amount ranging from 2% by wt. to 10% by wt. and (c) one or more excipients. In an embodiment, the essential oil includes spearmint oil, eucalyptus oil, peppermint oil, clove bud oil, thyme oil, capsicum oleoresin oil, lavender oil, rosemary oil, myrrh oil, ginger oil, orange sweet oil, chamomile oil, wintergreen oil, ajwain oil and combinations thereof. In an embodiment, the excipient includes: an emulsifier, a pH modifying agent, a solvent, a gelling agent and combinations thereof. In an embodiment, the active ingredient includes: an extract of chaste tree, an extract of guggul, an extract of triglyphix sense, menthol, camphor and combinations thereof. In an embodiment, the gelling agent includes carbomer. In an embodiment, the topical formulation is a cream formulation.

In an embodiment, the cream formulation further includes a particulate stock formulation in an amount ranging from 5% by wt. to 75% by wt., said stock formulation including: a natural analgesic, a lipid matrix forming agent, a permeability enhancer, a solubilizer, a surfactant and a solvent, wherein said natural analgesic is different from said active ingredient and said essential oil. In an embodiment, wherein said natural analgesic may also possess an anti-inflammatory activity.

In an embodiment, the topical formulation includes the particulate stock formulation in an amount ranging from 10% by wt. to 70% by wt., preferably, in an amount ranging from 20% by wt. to 60% by wt. and more preferably, in an amount ranging from 25% by wt. to 50% by wt. In an embodiment, the particulate stock formulation is a solid lipid nanoparticulate stock formulation. In an embodiment, the solid lipid nanoparticulate (SLN) has a particle size ranging from 10 and 1000 nanometers. In another embodiment, the solid lipid nanoparticulate has a particle size ranging from 50 and 1000 nanometers.

In an embodiment, said stock formulation includes: the natural analgesic in an amount ranging from 1% to 25% by wt.; the lipid matrix forming agent in an amount ranging from 3% to 20% by wt., preferably, 3-5% by wt.; the permeability enhancer in an amount ranging from 2% to 15% by wt., preferably, 2-5% by wt.; the solubilizer in an amount ranging from 5% to 25% by wt., preferably, 5-10% by wt.; the surfactant in an amount ranging from 5% to 25% by wt., preferably, 5-10% by wt.; and the solvent in an amount ranging from 5% to 25% by wt., preferably, 8-15% by wt.

In an embodiment, the lipid matrix forming agent includes Cholesterol, Precirol® ATO, Span, Phospholipid or combinations thereof.

In an embodiment, the permeability enhancer includes Basil oil, Eucalyptus oil, Menthol or combinations thereof.

In an embodiment, the solubilizer includes Polyethylene glycol, Propylene glycol or combinations thereof.

In an embodiment, the surfactant includes Transcutol, Tween or combinations thereof.

In an embodiment, the solvent is water.

In an embodiment, the natural analgesic is selected from any or a combination of: a cannabinoid concentrate, a cannabinoid isolate, Curcuma longa, Boswellia serrat, Symphytum omcinale, Hypericum perforatum, Ruta graveolens, white willow, fish oil or combinations thereof.

In an embodiment, the cream formulation has firmness (peak positive force at cycle 1) ranging from 140 g to 180 g.

In an embodiment, the cream formulation has stickiness (peak negative force at cycle 1) ranging from −150 g to −190 g.

In an embodiment, the cream formulation has work of shear (Area F-T 1:2) ranging from 160 g·sec to 190 g·sec.

In an embodiment, the cream formulation has an index of viscosity (Area F-T 3:4) ranging from −20 g·sec to −50 g·sec.

In an embodiment, the cream formulation has a penetration rate (flux) of at least 12 μg/(cm2*h) when measured using Franz diffusion cell mounted with a Strat-M membrane.

In an embodiment, the cream formulation has a penetration rate (flux) of at least 15 μg/(cm2*h) when measured using Franz diffusion cell mounted with a Strat-M membrane.

In an embodiment, the cream formulation has a penetration rate (flux) of at least 20 μg/(cm2*h) when measured using Franz diffusion cell mounted with a Strat-M membrane.

In an embodiment, the cream formulation has a penetration rate (flux) ranging from 20 to 27 μg/(cm2*h) when measured using Franz diffusion cell mounted with a Strat-M membrane.

In an embodiment, the cream formulation has ex-vivo skin penetration of at least 200 μm when measured on Guinea pig skin using confocal microscopy, preferably, at least 250 μm, and more preferably, at least 300 μm.

In an embodiment, the cream formulation possesses analgesic and anti-inflammatory properties.

In an embodiment, a method of reducing pain and inflammation in a mammal resulting from muscle strain, spasms, arthritis, and bursitis associated with sports related injury is by topical administration of the formulation.

In an embodiment, a method of reducing pain and inflammation in a mammal resulting from muscle strain, spasms, arthritis, and bursitis associated with sports related injury is by topical administration of the cream formulation.

Another aspect of the present disclosure relates to a method of preparation of a cream formulation, said method including the steps of: (a) taking essential oils and oil soluble ingredients to preparing an oil phase; (b) mixing active ingredient(s) and water soluble ingredients with water to prepare an aqueous solution; (c) mixing a gelling agent with the aqueous solution to prepare a water phase; (d) mixing a particulate stock formulation with the water phase to prepare a mixture; (e) adding the oil phase to the mixture from step (d) with stirring to prepare a crude emulsion; (f) adding an emulsifying agent and a pH modifying agent to the crude emulsion to prepare a coarse emulsion; and (g) homogenizing the coarse emulsion to prepare the cream formulation. In an embodiment, the particulate stock formulation is a solid lipid nanoparticulate stock formulation.

In an embodiment, the essential oil and oil soluble ingredients includes spearmint oil, eucalyptus oil, peppermint oil, clove bud oil, thyme oil, capsicum oleoresin oil, lavender oil, rosemary oil, myrrh oil, ginger oil, orange sweet oil, chamomile oil, wintergreen oil, ajwain oil, Camphor, Cetyl alcohol, Arlacel and combinations thereof.

In an embodiment, the active ingredient(s) and water soluble ingredients includes Triglyphix Sense, water extract of Guggul, water extract of Vitexnegundo and combinations thereof.

In an embodiment, the emulsifying agent is Arlacel, Tween 80, Cetyl alcohol, cocomonoethanolamide or Glyceryl monosterate. In an embodiment, the pH modifying agent is selected from NaOH, KOH or Citric acid.

In an embodiment, the solid lipid nanoparticulate stock formulation is prepared by: (a) preparing a lipid phase by mixing at least a part of a lipid matrix forming agent with a permeability enhancer; (b) preparing a natural analgesic phase by mixing the natural analgesic and optionally, a part of a lipid matrix forming agent with a solubilizer; (c) preparing a natural analgesic lipid phase by mixing the natural analgesic phase with the lipid phase; (d) preparing a surfactant solution by mixing a surfactant with a solvent; (e) mixing the natural analgesic lipid phase, maintained at a temperature ranging from 40° C. to 70° C., with the surfactant solution, maintained at a temperature ranging from 40° C. to 70° C., to obtain a mixture; and (e) subjecting the mixture obtained from step (e) to homogenization to obtain the solid lipid nanoparticulate stock formulation.

In an embodiment, the lipid matrix forming agent includes a combination of Cholesterol, Precirol® ATO, Span, and Phospholipid; the permeability enhancer includes a combination of Basil oil, Eucalyptus oil and Menthol, the solubilizer includes a combination of Polyethylene glycol and Propylene glycol, the surfactant includes a combination of Transcutol and Tween and the solvent is water.

In an embodiment, the step of homogenization is done for a time period ranging from 1 minute to 45 minutes at a speed ranging from 5000 rpm to 50000 rpm.

### LIST OF EMBODIMENTS

1. A topical formulation comprising:
(a) an essential oil in an amount ranging from 10% by wt. to 45% by wt.;
(b) an active ingredient in an amount ranging from 2% by wt. to 10% by wt. and
(c) an excipient.

2. The formulation as mentioned in item 1, wherein the essential oil comprises spearmint oil, eucalyptus oil, peppermint oil, clove bud oil, thyme oil, capsicum oleoresin oil, lavender oil, rosemary oil, myrrh oil, ginger oil, orange sweet oil, chamomile oil, wintergreen oil, ajwain oil and combinations thereof.

3. The formulation as mentioned in item 1, wherein the excipient comprises: an emulsifier, a pH modifying agent, a solvent, a gelling agent and combinations thereof.

4. The formulation as mentioned in item 1, wherein the active ingredient comprises: an extract of chaste tree, an extract of guggul, an extract of triglyphix sense, menthol, camphor and combinations thereof.

5. The formulation as mentioned in item 1, wherein the topical formulation is a cream formulation.

6. The formulation as mentioned in item 5, wherein the cream formulation has firmness (peak positive force at cycle 1) ranging from 140 g to 180 g.

7. The formulation as mentioned in item 5, wherein the cream formulation has stickiness (peak negative force at cycle 1) ranging from −150 g to −190 g.

8. The formulation as mentioned in item 5, wherein the cream formulation has work of shear (Area F-T 1:2) ranging from 160 g·sec to 190 g·sec.

9. The formulation as mentioned in item 5, wherein the cream formulation has index of viscosity (Area F-T 3:4) ranging from −20 g·sec to −50 g·sec.

10. The formulation as mentioned in item 5, wherein the cream formulation has a penetration rate (flux) of at least 12 μg/(cm2*h) when measured using Franz diffusion cell mounted with a Strat-M membrane.

11. The formulation as mentioned in item 5, wherein the cream formulation has a penetration rate (flux) of at least 15 μg/(cm2*h) when measured using Franz diffusion cell mounted with a Strat-M membrane.

12. The formulation as mentioned in item 5, wherein the cream formulation has a penetration rate (flux) of at least 20 μg/(cm2*h) when measured using Franz diffusion cell mounted with a Strat-M membrane.

13. The formulation as mentioned in item 5, wherein the cream formulation has a penetration rate (flux) ranging from 20 to 27 μg/(cm2*h) when measured using Franz diffusion cell mounted with a Strat-M membrane.

14. The formulation as mentioned in item 5, wherein the cream formulation has ex-vivo skin penetration of at least 200 μm when measured on Guinea pig skin using confocal microscopy, preferably, at least 250 μm, and more preferably, at least 300 μm.

15. The formulation as mentioned in item 5, wherein the cream formulation further comprises a particulate stock formulation in an amount ranging from 5% by wt. to 75% by wt., said stock formulation comprising: a natural analgesic, a lipid matrix forming agent, a permeability enhancer, a solubilizer, a surfactant and a solvent, wherein said natural analgesic is different from said active ingredient and said essential oil.

16. The formulation as mentioned in item 15, wherein the particulate stock formulation is a solid lipid nanoparticulate stock formulation.

17. A method of preparation of a cream formulation, said method comprising the steps of:
(a) taking essential oils and oil soluble ingredients to preparing an oil phase;
(b) mixing active ingredient(s) and water soluble ingredients with water to prepare an aqueous solution;
(c) mixing a gelling agent with the aqueous solution to prepare a water phase;
(d) mixing a particulate stock formulation with the water phase to prepare a mixture;
(e) adding the oil phase to the mixture from step (d) with stirring to prepare a crude emulsion;
(f) adding an emulsifying agent and a pH modifying agent to the crude emulsion to prepare a coarse emulsion; and
(g) homogenizing the coarse emulsion to prepare the cream formulation.

While the foregoing description discloses various embodiments of the disclosure, other and further embodiments of the invention may be devised without departing from the basic scope of the disclosure. The invention is not limited to the described embodiments, versions or examples, which are included to enable a person having ordinary skill in the art to make and use the invention when combined with information and knowledge available to the person having ordinary skill in the art.

### EXAMPLES

### A Cream Formulation

TABLE 1
A Cream Formulation

| | | AMOUNT |
|---|---|---|
| S. NO | INGREDIENTS | (in wt. %) |
| 1 | Spearmint Oil | 2 |
| 2 | Eucalyptus oil | 5 |
| 3 | Peppermint oil | 5 |
| 4 | Camphor | 3.1 |
| 5 | Clove bud oil | 1 |
| 6 | Thyme oil | 3 |
| 7 | Capsicum oleoresin oil | 0.1 |
| 8 | Lavender oil | 1 |
| 9 | Rosemary oil | 1 |
| 10 | Myrrh oil | 0.5 |
| 11 | Ginger oil | 0.5 |
| 12 | Orange Sweet oil | 0.5 |
| 13 | Chamomile oil | 0.5 |
| 14 | Wintergreen oil | 5 |
| 15 | Ajwain oil | 0.5 |
| 16 | Cetyl alcohol | 2 |
| 17 | Arlacel | 1 |
| 18 | Vitex negundo water extract | 0.5 |
| 19 | Guggul water extract | 0.5 |
| 20 | Trigliphix Sense | 0.5 |
| 21 | Carbopol 940 | 1 |
| 22 | Solid lipid nanoparticulate stock | 40 |
| | formulation | |
| 23 | Water | 24.9 |
| 24 | Tween 80 | 1 |
| 25 | NaOH | q.s. |

| | |
|---|---|
| Total wt. % | 100 |

TABLE 1A
Another embodiment of a Cream Formulation

| | | AMOUNT |
|---|---|---|
| S. NO | INGREDIENTS | (in wt. %) |
| 1 | Spearmint Oil | 2 |
| 2 | Eucalyptus oil | 5 |
| 3 | Peppermint oil | 5 |
| 4 | Camphor | 3.1 |
| 5 | Clove bud oil | 1 |
| 6 | Thyme oil | 2 |
| 7 | Capsicum oleoresin oil | 0.1 |
| 8 | Lavender oil | 1 |
| 9 | Rosemary oil | 1 |
| 10 | Myrrh oil | 0.5 |
| 11 | Ginger oil | 0.5 |
| 12 | Orange Sweet oil | 0.5 |
| 13 | Chamomile oil | 0.5 |
| 14 | Wintergreen oil | 5 |
| 15 | Ajwain oil | 0.5 |
| 16 | Cetyl alcohol | 2 |
| 17 | Arlacel | 1 |
| 18 | Vitex negundo water extract | 0.5 |
| 19 | Guggul water extract | 0.5 |
| 20 | Triglyphix Sense | 0.5 |
| 21 | Carbopol 940 | 0.5 |
| 22 | Solid lipid nanoparticulate stock | 40 |
| | formulation | |
| 23 | Water | 24.3 |
| 24 | Tween 80 | 1 |
| 25 | NaOH | q.s. |

| | |
|---|---|
| Total wt. % | 100 |

All the ingredients were weighed accurately as per Table 1 above. The method of preparation of a cream formulation includes steps as follow:

### Step 1—Preparation of an Oil Phase

All the oil based ingredients namely Spearmint Oil, Eucalyptus oil, Peppermint oil, Camphor, Clove bud oil, Thyme oil, Capsicum oleoresin oil, Lavender oil, Rosemary oil, Myrrh oil, Ginger oil, Orange Sweet oil, Chamomile oil, Wintergreen oil, Ajwain oil, Cetyl alcohol and Arlacel were taken in a beaker and heated between 25 to ≤40° C. slightly to form an oil phase.

### Step 2—Preparation of an Aqueous Phase

All the active ingredient(s) aqueous Triglyphix Sense and water extracts of Guggul and Vitexnegundo were taken in a beaker and mixed with water at room temperature to prepare a solution. A gelling agent carbomer was added into the above solution to obtain a mixture. Solid lipid nanoparticulate stock formulation was taken and added into the above prepared mixture to obtain an aqueous phase.

### Step 3—Preparation of Emulsion

Above prepared oil phase was added into the aqueous phase under stirring to obtain a crude emulsion. An emulsifying agent (Tween 80) and a pH modifying agent (NaOH or KOH) were added into the crude emulsion to obtain a coarse emulsion and to keep the pH of the cream between 5.0 and 6.0. This coarse emulsion was homogenized at 10000 RPM for 15 minutes to obtain a cream formulation.

Composition for a solid lipid nanoparticulate stock formulation used in the cream formulation is provided in the Table 2 below:

TABLE 2
Composition for solid lipid nanoparticulate
(SLN) stock formulation

| PHASE | INGREDIENTS | QUANTITY | |
|---|---|---|---|
| Lipid Phase | Cholesterol | 150 | mg |
| | Precirol ATO | 150 | mg |
| | Span 60 | 4 | g |
| | Basil oil | 1 | ml |
| | Eucalyptus oil | 1 | ml |
| | Menthol | 1 | g |
| Natural analgesic | Cannabidiol concentrate | 6 | g |
| Phase | Cannabidiol Isolate | 2 | g |
| | Phospholipid | 100 | mg |
| | Polyethylene glycol 400 | 4 | ml |
| | Propylene glycol | 4 | ml |
| Surfactant Solution | Transcutol P | 4 | ml |
| | Tween 80 | 4 | ml |
| | Water | 8.6 | ml |

TABLE 2A
Another embodiment of composition for solid
lipid nanoparticulate (SLN) stock formulation

| PHASE | INGREDIENTS | QUANTITY | |
|---|---|---|---|
| Lipid Phase | Cholesterol | 150 | mg |
| | Precirol ATO | 150 | mg |
| | Span 60 | 4 | g |
| | Basil oil | 1 | ml |
| | Eucalyptus oil | 1 | ml |
| | Menthol | 1 | g |
| Natural analgesic | Cannabidiol concentrate | 3.73 | g |
| Phase | Cannabidiol Isolate | 2 | g |
| | Phospholipid | 100 | mg |
| | Polyethylene glycol 400 | 4 | ml |
| | Propylene glycol | 4 | ml |
| Surfactant Solution | Transcutol P | 4 | ml |
| | Tween 80 | 4 | ml |
| | Water | 10.87 | ml |

The solid lipid nanoparticulate stock formulation was prepared according to below-mentioned process:

### Step 1—Preparation of Lipid Phase

Cholesterol, precirol ATO and span 60 were taken and heated to a temperature between 25 to ≤40° C. to obtain a molten mixture. In the molten mixture, basil oil, eucalyptus oil and menthol were added and mixed thoroughly to obtain a lipid phase.

Step 2—Preparation of Natural analgesic Phase

Natural analgesic(s) (viz. Cannabinoidconcentrate and cannabinoid isolate) and phospholipid were taken and dissolved in a mixture of propylene glycol and polyethylene glycol 400 by using ultrasonic bath sonicator. The mixture was slightly heated between 25 to 40° C. to obtain a natural analgesic phase.

Step 3—Preparation of natural analgesic Lipid Phase

Above prepared natural analgesic phase was added to the lipid phase under constant stirring and mild heating between 25 to 40° C. to obtain a natural analgesic lipid phase.

### Step 4—Preparation of Surfactant Solution

Tween 80 and Transcutol P were mixed together in water to obtain a surfactant solution.

Step 5—Mixing of natural analgesic Lipid Phase and surfactant solution

Bothnatural analgesic lipid phase and surfactant solution were maintained at 60° C. and then natural analgesic lipid phase was added into the surfactant solution to obtain a mixture. This mixture was homogenized at 15,000 rpm for about 15 min to obtain a solid lipid nanoparticulate stock formulation.

The cream formulation prepared above was subjected to texture analysis to understand its characteristics vis-à-vis that of commercially available cream formulation moov, results whereof are provided in the Table 3 below.

TABLE 3
Texture analysis of cream formulation and its comparison
with a standard cream formulation (moov)

| | | Stickiness (g) | | Index of |
|---|---|---|---|---|
| | Firmness (g) | Peak Negative | Work of shear | viscosity |
| | Peak Positive | Force | (g · sec) | (g · sec) |
| Test ID | Force (Cycle: 1) | (Cycle: 1) | Area F-T 1:2 | Area F-T 3:4 |
| Standard Cream | 915.239 | −875.348 | 1748.217 | −311.74 |
| Formulation | | | | |
| (Moov) | | | | |
| Cream | 161.814 | −173.299 | 174.411 | −34.329 |
| Formulation | | | | |

### Permeability Assessment In Vitro

Permeability assessment of cream formulation and other standard formulations were performed by in vitro permeation through Strat-M membranes. Strat-M membrane was mounted on a Franz diffusion cell. Receptor media (PBS: Ethanol 7:3) was added in the receptor compartment. To avoid air bubbles formation receptor media was initially sonicated for about 15 min. The receptor media was stirred continuously during the experiment and temperature of the receptor media was maintained at 37° C. The absence of air bubble was monitored throughout the experiment. 200 mg of formulations was applied on Strat-M membrane in the donor compartment. Parafilm® (Semi-occlusive) covered the opening of the Franz Diffusion Cell. 100 μl of formulations were withdrawn from receptor compartment at 0, 1, 2, 3, 4, 5, 6, 7 & 8 h, simultaneously equal volume of blank receptor media was added to compensate the withdrawn volume. Experiments were performed in triplicates for each formulation.

### Penetration Assessment Ex Vivo

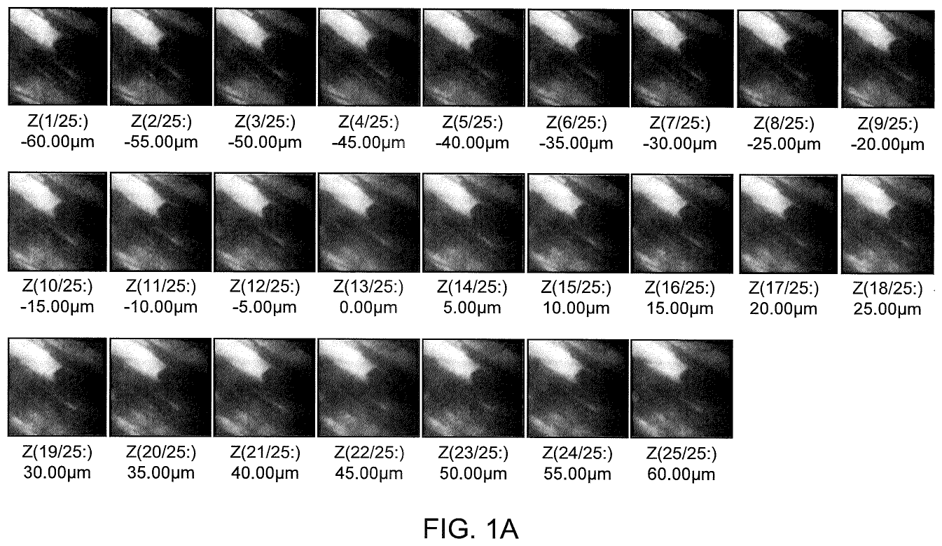

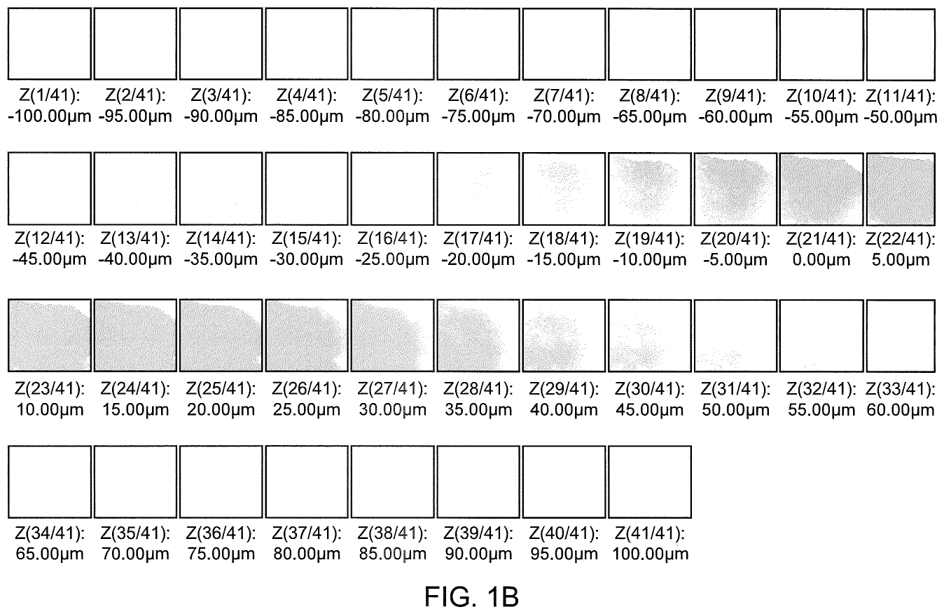

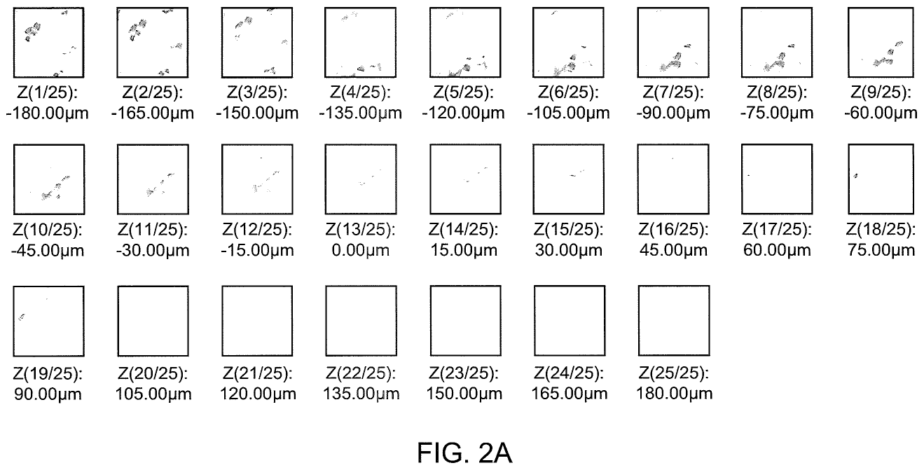

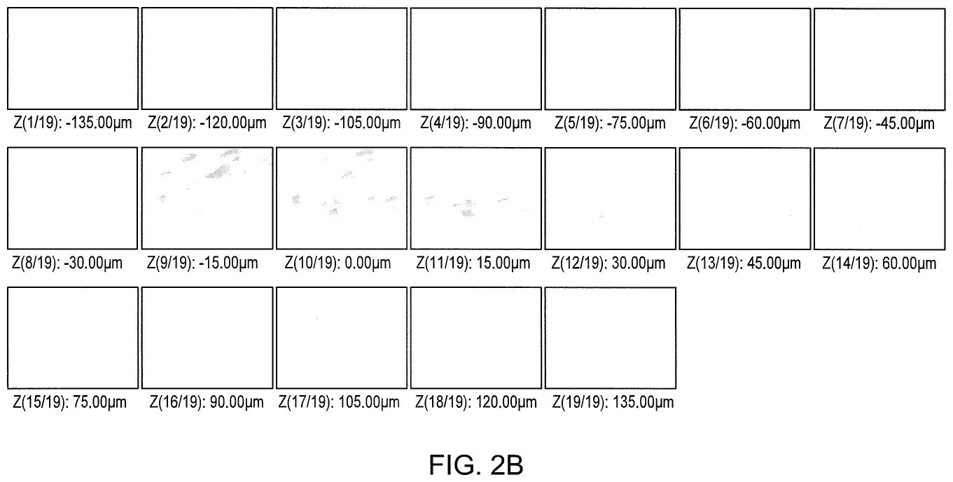

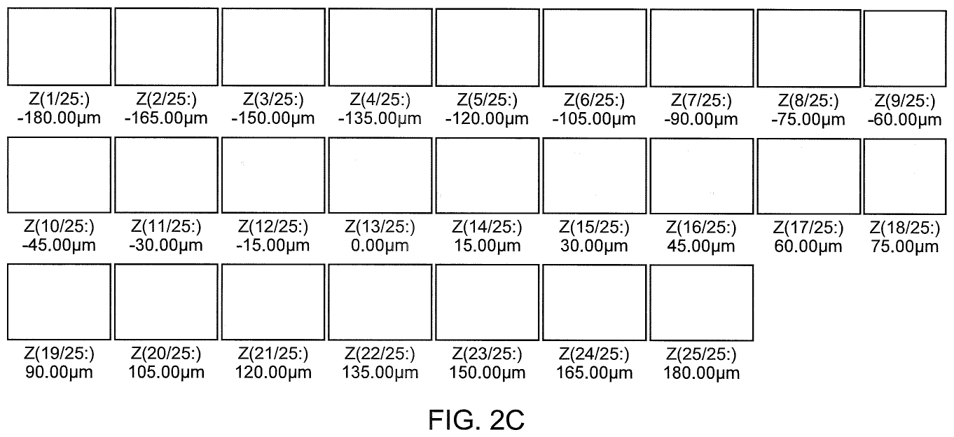

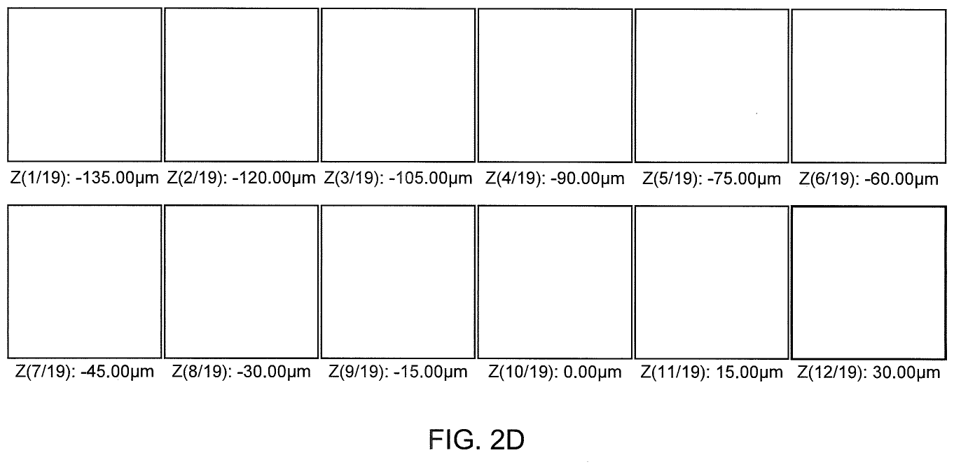

Penetration assessment of cream formulation and other standard formulations were performed using excised rat and pig skin by Confocal imaging. To corroborate the drug delivery from the cream formulation into a distinguished layer of skin, a fluorescent dye rhodamine B was used to predict the distribution of drug using this technique. FIGS. 1A and 1B show the confocal microscopy of rat skin with z-stack (penetration depth profile) image at scanning rate of 5 μm across skin layers, showing distribution of rhodamine fluorescence particle from rhodamine solution up to depth of 20 μm for placebo and 185 μm for cream formulation, respectively. FIG. 2A through 2D show the confocal microscopy of guinea pig skin with z-stack (penetration depth profile) image at scanning rate of 15 μm across skin layer, showing distribution of rhodamine fluorescence particle from rhodamine solution up to depth of 330 μm for the cream formulation prepared above, 150 μm for Charlotte's web, 135 μm for CBD Medic and 60 μm for Myaderm.

### Efficacy Assessments of Cream Formulation

### Anti-Inflammatory Activity

COX-2 is an enzyme responsible for inflammation and pain. Inhibition of COX-2 plays a key role in analgesic action.

### Comparison of Anti-Inflammatory Activity of the Cream Formulation

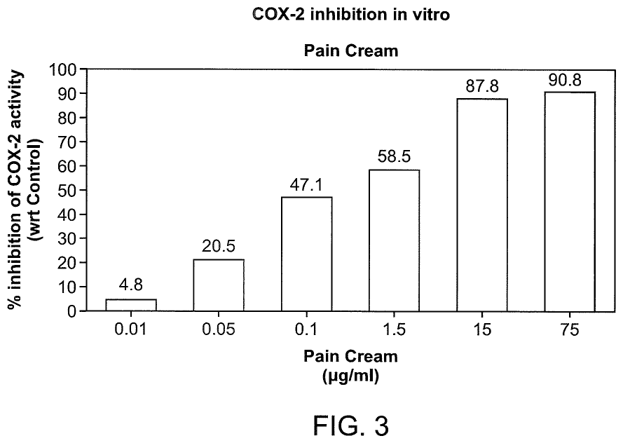

The cream formulation was dissolved in DMSO to prepare a 500 mg/ml stock solution and was diluted to achieve final test concentrations of 0.01 to 75 μg/ml. COX-2 enzyme was incubated with different concentrations of cream formulation to determine % inhibition of COX-2 activity and IC₅₀ value. FIG. 3 illustrates a graph depicting % inhibition of COX-2 activity for the cream formulation. The cream formulation demonstrated analgesic effect by in vitro inhibition of COX-2 enzymatic activity having IC50 value of only 0.4367 μg/ml.

### Comparison of Anti-Inflammatory Activity of Cream Formulation and Standard Formulations (Myaderm, Charlott's Web and CBD Medic)

Anti-inflammatory activity of cream formulation was measured using carrageenan-induced rat paw edema model. Healthy adult wistar rats, (8-10 weeks old) were enrolled in the experiment. Animals were allocated to various groups and each group consisted of six rats. Basal paw thickness was measured using a digital micrometer (Mitutoyo, Japan). Rats were injected with 0.1 ml λ-Carrageenan (1% in distilled water) into the left hind paw (sub-plantar injection). 200 mg of placebo, cream formulation & standard formulations (Myaderm, Charlott's Web and CBD Medic) were topically applied uniformly on the paw immediately after Carrageenan injection. Paw thickness was measured again at 30 min, 60 min, 120 min & 240 min after application of test formulations. % anti-inflammatory activity of cream formulation was calculated at each time point. Mean paw edema was calculated and % anti-inflammatory activity was also calculated when compared to basal measurement and subjected to Bonferroni's test multiple comparison test.

TABLE 4
Effect of cream formulation and standard
formulations on Inflammatory Paw edema

| | Mean Paw Edema (cm.) | | | | |
|---|---|---|---|---|---|
| Treatment | | 30 min. | 60 min. | 120 min. | 240 min. |
| Placebo | Mean | 1.87 | 2.14 | 2.28 | 2.48 |
| Control | SEM | 0.17 | 9.09 | 0.09 | 0.18 |
| Cream | Mean | 1.41 | 1.58 | 3.77 | 2.27 |
| Formulation, | SEM | 0.25 | 0 25 | 0.22 | 0.19 |
| 200 mg | | | | | |
| Myaderm, | Mean | 1.68 | 1.87 | 1.94 | 2.25 |
| 200 mg | SEM | 0.17 | 0.09 | 0.17 | 0.14 |
| Charlott's Web, | Mean | 1.77 | 1.82 | 1.74 | 2.20 |
| 200 mg | SEM | 0.18 | 0.18 | 0.18 | 0.30 |
| CBD Medic, | Mean | 3.84 | 1.63 | 3.75 | 2.34 |
| 200 mg | SEM | 0.06 | 0.11 | 0.11 | 0.10 |

TABLE 5
% Anti-inflammatory activity of cream formulation and
other standard formulations as compared to placebo

| | % Anti-inflammatory Activity | | | |
|---|---|---|---|---|
| Treatment | 30 min. | 60 min. | 120 min. | 240 min. |
| Cream Formulation, 200 mg | 24.3 | 29.4 | 22.3 | 7.5 |
| Myaderm, 200 mg | 10.0 | 13.0 | 14.9 | 8.5 |
| Charlott's Web, 200 mg | 5.4 | 24.8 | 23.8 | 10.3 |
| CBD Medic, 200 mg | 1.4 | 24.2 | 23.4 | 13.1 |

Topical application of cream formulation demonstrated considerable anti-inflammatory activity in the carrageenan-induced rat paw edema model at the given dose of 200 mg at 30, 60 and 120 minutes as compared to placebo control (24.3, 29.4 & 22.3% respectively).

### Analgesic Activity

Analgesic activity of cream formulation was measured using rat tail flick method. The method used in this study was the tail flick test with radiant heat. Tail flick analgesia meter was used for this test. The equipment allows exposure of the middle portion of the tail of the rat to a point source of radiant heat. The rat withdraws its tail in response to the applied heat. The reaction time of the phenomena was known as tail flick latency. A prolonged reaction time was considered as an analgesic activity. A cut-off time of 40 seconds was set as a threshold to avoid damage to the tail of the animal. Healthy adult male wistar rats (8-10 weeks) were enrolled in the experiment. Animals were allocated to various groups and each group consisted of six rats. 200 mg of Placebo, cream formulation & standard formulations (Myaderm, Charlott's Web and CBD Medic) were topically applied uniformly at the middle portion of the tail of each rat. The tail flick latency was recorded using the above mentioned tail flick apparatus prior to application (0 min) and at 30, 60, 120 & 240 min after application of various formulations. Mean reaction time in seconds were taken, mean was calculated, % activity was also calculated when compared to Placebo and subjected to Bonferroni's test multiple comparison test.

TABLE 6
Tail Flick reactions of cream formulation
and other standard formulations

| | | Tall Flick Reaction (Time In Second) | | | | |
|---|---|---|---|---|---|---|
| Treatment Groups | | 0 min | 30 min | 60 min | 120 min | 240 min |
| Placebo, 200 mg | Mean | 15.3 | 15.7 | 15.6 | 15.4 | 15.2 |
| | SEM | 0.56 | 0.34 | 0.49 | 0.45 | 0.66 |
| Cream Formulation, | Mean | 15.0 | 29.3 | 27.3 | 23.5 | 19.0 |
| 200 mg | SEM | 0.62 | 3.03 | 2.37 | 2.19 | 1.25 |
| Myaderm, 200 mg | Mean | 14.2 | 25.6 | 20.1 | 17.3 | 16.5 |
| | SEM | 0.77 | 1.68 | 0.59 | 0.70 | 0.68 |
| Chorlette Web, | Mean | 14.5 | 27.4 | 23.7 | 19.2 | 17.1 |
| 200 mg | SEM | 1.45 | 1.02 | 0.54 | 0.88 | 0.60 |

TABLE 7
% Analgesic activity of cream formulation
and other standard formulations

| | % Activity | | | |
|---|---|---|---|---|
| Treatment Groups | 30 min | 60 min | 120 min | 240 min |
| Myaderm, 200 mg | 80.6 | 41.7 | 21.7 | 16.3 |
| Chorlette Web, 200 mg | 88.3 | 63.1 | 31.9 | 17.4 |
| Cream Formulation, 200 mg | 94.6 | 81.6 | 56.2 | 26.6 |

Topical application of cream formulation demonstrated significant anti-nociceptive/analgesic activity (p<0.01 & 0.5) in the tail flick test at the given dose of 200 mg at all the tested time points i.e. 30, 60, 120 and 240 min. as compared to placebo control (94.6%, 81.6%, 56.2% & 26.6% respectively).

We claim:

1. A topical cream formulation, comprising:
a) an oil phase comprising of one or more essential oils, one or more active ingredients, a first emulsifying agent and cetyl alcohol;
b) an aqueous phase comprising of one or more active ingredients, a gelling agent, a solid lipid nanoparticulate stock formulation and water;
c) a second emulsifying agent; and
d) a pH modifying agent.

2. A topical cream formulation according to claim 1, wherein the essential oil of the oil phase is selected from Spearmint Oil, Eucalyptus oil, Peppermint oil, Clove bud oil, Thyme oil, Capsicum oleoresin oil, Lavender oil, Rosemary oil, Myrrh oil, Ginger oil, Orange Sweet oil, Chamomile oil, Wintergreen oil, Ajwain oil or a combination thereof.

3. A topical cream formulation according to claim 1, wherein the active ingredient of the aqueous phase or oil phase is selected from Vitex negundo water extract, Guggul water extract, Triglyphix Sense extract, camphor, menthol or combinations thereof.

4. A topical cream formulation according to claim 1, wherein the first and second emulsifying agent is selected from Arlacel, Tween 80, Cetyl alcohol, cocomonoethanolamide, Glyceryl monosterate or combinations thereof.

5. A topical cream formulation according to claim 1, wherein the pH modifying agent is selected from NaOH, KOH, Citric acid, or combinations thereof.

6. A solid lipid nanoparticulate stock formulation for the preparation of a topical cream formulation, said solid lipid nanoparticulate stock formulation comprising:
a natural analgesic lipid phase comprising:
a lipid phase comprising one or more of a first lipid matrix forming agent and a permeability enhancer;
a natural analgesic phase comprising one or more of a natural analgesic, a second lipid matrix forming agent and a solubilizer; and
a surfactant solution comprising a surfactant and water.

7. A solid lipid nanoparticulate stock formulation according to claim 6, wherein the first or second lipid matrix forming agent of the lipid phase is selected from Cholesterol, Precirol® ATO, Span, Phospholipid or combinations thereof.

8. A solid lipid nanoparticulate stock formulation according to claim 6, wherein the permeability enhancer is selected from Basil oil, Eucalyptus oil, Menthol or combinations thereof.

9. A solid lipid nanoparticulate stock formulation according to claim 6, wherein the natural analgesic of the natural analgesic phase is selected from a cannabinoid concentrate, a cannabinoid isolate, Curcuma longa, Boswellia serrat, Symphytum omcinale, Hypericum perforatum, Ruta graveolens, white willow, fish oil or combinations thereof.

10. A solid lipid nanoparticulate stock formulation according to claim 6, wherein the solubilizer is selected from Polyethylene glycol, Propylene glycol or combinations thereof.

11. A solid lipid nanoparticulate stock formulation according to claim 6, wherein the surfactant of the surfactant solution is selected from Tween 80, TranscutolP or combinations thereof.

12. A method for preparing a topical cream formulation comprising the steps of:
a) mixing one or more of essential oils, one or more active ingredients, a first emulsifying agent and cetyl alcohol to obtain a mixture;
b) heating the mixture of step a) between 25 to 40° C. to obtain the oil phase;
c) mixing one or more of active ingredients and water to obtain a solution;
d) adding a gelling agent and solid lipid nanoparticulate stock formulation to the solution of step c) to obtain the aqueous phase;
e) adding the oil phase of step b) to the aqueous phase of step d) to obtain a crude emulsion;
f) adding an emulsifying agent and a pH modifying agent to the crude emulsion of step e) to obtain a coarse emulsion; and
g) homogenising the coarse emulsion to obtain a topical cream formulation.

13. A method for preparing a topical cream formulation according to claim 12, wherein the pH modifying agent of step f) is added to maintain the pH of the topical cream between 5 and 6.

14. A method of preparing a topical cream formulation according to claim 12, wherein the homogenisation is carried out between 5,000 rpm to 50,000 rpm for a period of 1 minute to 45 minutes.

15. A method of reducing topical inflammation in a mammal in need thereof comprising topically administering to said mammal an amount effective to produce a topical anti-inflammatory effect of a composition comprising:
a) an oil phase comprising of one or more essential oils, at least a first emulsifying agent and cetyl alcohol;
b) an aqueous phase comprising of one or more active ingredients, a gelling agent, a solid lipid nanoparticulate stock formulation and water;
c) a second emulsifying agent; and
d) a pH modifying agent.

16. A method of reducing pain in a mammal in need thereof which comprises topically administering to said mammal an amount effective to produce a topical pain reducing effect of a composition comprising:
a) an oil phase comprising of one or more essential oils, at least a first emulsifying agent and cetyl alcohol;
b) an aqueous phase comprising of one or more active ingredients, a gelling agent, a solid lipid nanoparticulate stock formulation and water;
c) a second emulsifying agent; and
d) a pH modifying agent.
